(19) United States
(12) Patent Application Publication
(10) Pub. No.: US 2012/0321728 A1
(43) Pub. Date: Dec. 20, 2012

(54) NUTRITIONAL FORMULA FOR MANAGING ANGINA PECTORIS

(76) Inventor: Ven Subbiah, Garner, NC (US)

(21) Appl. No.: 13/492,973

(22) Filed: Jun. 11, 2012

Related U.S. Application Data
(60) Provisional application No. 61/497,098, filed on Jun. 15, 2011.

(51) Int. Cl. A61K 36/537; A61P 7/00

(52) U.S. Cl. 424/746

(57) ABSTRACT

The present invention is a novel nutritional product composition, formulated to be used as medical foods for managing blood flow related problems. The formula comprises clary sage root extract highly enriched for Tanshinones L-arginine, niacin, vitamin C, Vitamin B6, vitamin B12, folate and soy isoflavones. The composition enhances the increased blood flow to the muscle mass. Tanshinone IIA is an inducer of heme oxygenase 1 and suppressor of the induction of inducible nitric oxide synthase, which could result in desirable effects on two important gaseous signaling molecules, namely carbon monoxide and nitric oxide that have significant anti-inflammatory properties.

This application claims priority of U.S. provisional application No. 61/497,098 filed on Jun. 15, 2011 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention generally relates to dietary supplements, and more particularly to a medical food formula for improving blood flow comprising clary sage root extract, L-arginine, niacin, and vitamin C, such as in Angina pectoris and optionally containing Vitamin B6, vitamin B12, folate and soy isoflavones.

2. Description of Related Art

Angina pectoris is the medical term for chest pain or discomfort due to coronary heart disease. Angina is a symptom of a condition called myocardial ischemia. It occurs when the heart muscle (myocardium) doesn't get as much blood (hence as much oxygen) as it needs, i.e. poor blood flow to the area. Typical angina symptoms include uncomfortable pressure, fullness, squeezing or pain in the center of the chest. The discomfort also may be felt in the neck, jaw, shoulder, back or arm. Many types of chest discomfort aren't related to angina. Acid reflux (heartburn) and lung infection or inflammation are examples. Angina is a sign that someone is at increased risk of heart attack, cardiac arrest, and sudden cardiac death.

There are different types of Angina, i) Stable angina, also known as effort angina, refers to the more common understanding of angina related to myocardial ischemia. Typical presentations of stable angina is that of chest discomfort and associated symptoms precipitated by some activity (running, walking, etc.) with minimal or non-existent symptoms at rest.

ii) Unstable angina (also “crescendo angina) is a form of acute coronary syndrome is defined as angina pectoris that changes or worsens. It has at least one of these three features: a) it occurs at rest (or with minimal exertion), usually lasting >10 min; b) it is severe and of new onset (i.e., within the prior 4-6 weeks); and/or c) it occurs with a crescendo pattern (i.e., distinctly more severe, prolonged, or frequent than previously).

iii) Microvascular Angina or Angina Syndrome X is characterized by angina-like chest pain, but has different causes. The cause of Microvascular Angina is unknown, but it appears to be the result of poor function in the tiny blood vessels of the heart, arms and legs.

Clary sage enriched for Tanshinones is a specially formulated medical food product, consisting primarily of a proprietary standardized extract of Clary sage a GRAS plant. Clary sage is cultivated mainly for the production of essential oil, sclareol, and sclareol derivatives used in food products. Traditionally, sage is used as a tonic, digestive, antiseptic, astringent, and antispasmodic. Clary sage has also been used in the treatment of cancer. The most active chemicals that impart antioxidant and anti-inflammatory properties to Clary sage roots (rhizome) are Tanshinones. Tanshinones are orange-red pigments initially reported from the rhizomes of Salvia miltiorrhiza Bunge (Dan-Shen). Dan-Shen has been used for centuries in traditional Chinese medicine for the treatment of coronary heart disease, particularly angina pectoris and myocardial infarction (Adams JD et al. 2006, Preclinical and clinical examinations of Salvia miltiorrhiza and its tanshinones in ischemic conditions, Chin Med Nov, 23:1-3). Tanshinones have common structural features of planar phenanthrene quinine that are comprised of four compounds, Tanshinone I, Tanshinone IIA, Tanshinone IIB and Cryptotanshinone.

### BRIEF SUMMARY OF THE INVENTION

In accordance with an embodiment of the present invention, there is provided a food formula for use as a nutritional supplement, treatment or management of Angina Pectoris improved blood flow and muscle mass and exercise performance health.

One embodiment of the present invention is a composition for use in improving blood flow in a patient formulated for oral administration comprising: from 1 mg. to 3,000 mg. of clary sage root extract enriched with Tanshinones, particulary, Tanshinone II A;

from 1 mcg. to 2,000 mcg. of L-arginie;

from 1 mcg. to 1,000 mcg. of Niacin; and

from 1 mcg. to 1,000 mcg. of Vitamin C.

Another embodiment of the present invention is a method of improving blood flow in a patient in need thereof, comprising administering to the patient a composition comprising:

from 1 mg. to 3,000 mg. of clary sage root extract enriched with Tanshinones, particularly, Tanshinone II A;

from 1 mcg. to 2,000 mcg. of L-arginine;

from 1 mcg. to 1,000 mcg. of Niacin; and

from 1 mcg. to 1,000 mcg. of Vitamin C.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definitions

The terms “about” and “essentially” mean ±10 percent.

The term “comprising” is not intended to limit inventions to only claiming the present invention with such comprising language. Any invention using the term comprising could be separated into one or more claims using “consisting” or “consisting of” claim language and is so intended.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

The present invention comprises of a formula for use as a dietary supplement, dietary ingredient, and medical food. The formula is intended for use as a nutritional supplement, Angina Pectoris and as a muscle mass agent for increased blood flow in a selected patient. The manner in which the formula provides the specific effects will be discussed in greater detail below. In one embodiment the formula comprises 250 mg of clary sage root extract, 1.6 g L-arginine, 25 mg niacin 150 mg, vitamin C, 100 IU, 1 mg Vitamin B6, 2.4 microgram vitamin B12, 200 microgram folate and 20 mg soy isoflavones. The present formula is in one embodiment provided in Food bar, tablet, or capsule form though it will be appreciated that such may also be provided in powder or liquid form. For the desired results, the proper dosages are from one (1) to five (5) food bars or beverage of 2 ounces daily.

Ingredients in the present invention are from 1 mg. to 3,000 mg. of clary sage root extract enriched with Tanshinones, particularly, Tanshinone II A; from 1 mcg. to 2,000 mcg. of L-arginine; from 1 mcg. to 1,000 mcg. of Niacin; and from 1 mcg. to 1,000 mcg. of Vitamin C.

Optional ingredients include 1 to 200 IU of Vitamin E, 1 mg-100 mg of Vitamin B6, 1-100 micrograms of Vitamin B12, 1-100 mg of folate and 1-100 mg of soy isoflavones.

Tanshinone IIA is an inducer of heme oxygenase 1 and suppressor of the induction of inducible nitric oxide synthase, which could result in desirable effects on two important gaseous signaling molecules, namely carbon monoxide and nitric oxide that have significant anti-inflammatory properties.

L-arginine, niacin, Vitamin C, Vitamin E, Vitamin B6, Vitamin B12, Folate and Soy isoflavone. Components increased production of free radicals under oxidative stress conditions plays a vital role in the impairment of endothelial function and also in the pathogenesis, leads to the exacerbated formation of oxy-free radicals. These reactive oxygen species through a chain of reactions damage the cardiomyocytes and cause more injury to the lungs. L-Arginine is reported to act as free radical scavenger, inhibits the activity of pro-oxidant enzymes, and thus acts as an antioxidant and these roles of L-arginine are mediated by nitric oxide (NO). The effect of oral administration of L-arginine on some antioxidant enzymes, total thiols, lipid peroxidation measured as malondialdehyde (MDA), and plasma ascorbate levels. An increase in the activity of superoxide dismutase (SOD), total thiols (T-SH) and plasma ascorbate levels and a decrease in the activity of xanthine oxidase (XO), MDA levels, carbonyl content and serum cholesterol in the patients on oral administration of L-arginine. L-arginine dependent coronary segment was augmented by the antioxidant vitamin C in patients. Thus, vitamin C may have beneficial effects on nitric oxide bioavailability induced by L-arginine.

Folic acid combined with vitamins B (6) and B (12) is effective in lowering homocysteine levels. The effect of a folic acid-based supplement on secondary prevention of clinical events. As previously indicated, niacin is incorporated into the present formula primarily for purposes of improving the efficiency. Protein-calorie malnutrition is prevalent among individuals with Angina pectoris and suboptimal body weight has been associated with increased morbidity and mortality. Intake levels of niacin and vitamin C were adequate in terms of the RDAs and were not related to relative body weight. The inverse associations between dietary isoflavone and the prevalence of Angina pectoris are well documented. Diet, with a high intake of soy foods may be beneficial to muscle mass development.

Example of Clary Sage Formulation (Salvia sclarea) Enriched for Tanshinone

Clary sage roots were cut into small pieces and were dried in a drying oven at 45 degrees C. to remove all traces of moisture. The dried material was stored at −20 degrees C. before processing. Dried samples were pulverized using a Wiley Mill with fillers to obtain a 20-40 micron particle size. Samples were stored at −20 degrees C. in air tight ziplock bags.

The sample was systematically extracted using hexane and the hexane fractional washed with 5 percent sodium carbonate. Solvents were then removed with a rotating evaporator. The isolated fraction tests high for Tanshinones (designated SP001). Fingerprinting confirmed analysis using LC-MS. The enriched clary sage fraction was re-chromotographed on a hypersil C18 reversed phase column (100×2.1 mm, 5 micrometers) eluted with water-acetone gradient with a flow rate of 0.6 ml/min. Sample was monitored based on the UV character as well as micro detection. The LC/MS data was used to quantify Tanshinone IIA concentration in SP001.

### Example Dosage Formulation

The following formulation is an exemplary embodiment and formulated into capsules or food/candy bar type delivery means by methods known in the art. Clary Sage Root Extract enriched with Tanshinone: 250 mg Additional ingredients restore NO activity, facilitate vasodilation, and increase resistance to atherosclerosis and thrombosis.

| | | |
|---|---|---|
| L-arginine | 1.6 | g |
| Niacin | 25 | mg |
| Vitamin C | 150 | mg |
| Vitamin E | 100 | IU |
| Vitamin B6 | 1 | mg |
| Vitamin B12 | 2.4 | micro g |
| Folate | 0.200 | mg |
| Soy Isoflavones | 20 | mg |

Those skilled in the art to which the present invention pertains may make modifications resulting in other embodiments employing principles of the present invention without departing from its spirit or characteristics, particularly upon considering the foregoing teachings. Accordingly, the desired embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A composition for use in improving blood flow in a patient formulated for oral administration comprising:
from 1 mg. to 3,000 mg. of clary sage root extract enriched with Tanshinones, particularly, Tanshinone II A;
from 1 mcg. to 2,000 mcg. of L-arginine;
from 1 mcg. to 1,000 mcg. of Niacin; and
from 1 mcg. to 1,000 mcg. of Vitamin C.

2. The composition of claim 1 further comprising 1 to 200 IU of Vitamin E.

3. The composition of claim 1 further comprising 1 mg-100 mg. of Vitamin B6.

4. The composition of claim 1 further comprising 1-100 micrograms of Vitamin B12.

5. The composition of claim 1 further comprising 1-100 mg of Folate.

6. The composition of claim 1 further comprising 1-100 mg of Soy Isoflavones.

7. The composition according to claim 1 comprising:
250 mg. of clary sage root extract enriched with Tanshinones;
1.6 g. of L-arginine;
25 mg. of niacin;
150 mg. of Vitamin C;
100 IU of Vitamin E;
1 mg of Vitamin B6;
2.4 microgram of Vitamin B12;
200 microgram of Folate; and
20 mg Soy isoflavones.

8. The composition of claim 7 wherein the composition is formulated as a beverage.

9. The formula of claim 7 wherein the composition is formulated as a powder.

10. The formula of claim 7 wherein the composition is formulated as a liquid.

11. The formula of claim 7 wherein the composition is formulated as a food bar.

12. The formula of claim 7 wherein the composition is formulated as a tablet.

13. The composition of claim 1 formulated for use in treating Angina Pectoris agent.

14. A method of improving blood flow in a patient in need thereof, comprising administering to the patient a composition comprising:
from 1 mg. to 3,000 mg. of clary sage root extract enriched with Tanshinones, particularly, Tanshinone II A;
from 1 mcg. to 2,000 mcg. of L-arginine;
from 1 mcg. to 1,000 mcg. of Niacin; and
from 1 mcg. to 1,000 mcg. of Vitamin C.

15. The method according to claim 14, wherein the composition is administered twice daily.
